(19) United States
(12) Patent Application Publication
(10) Pub. No.: US 2015/0126573 A1
(43) Pub. Date: May 7, 2015

(54) METHODS AND PHARMACEUTICAL COMPOSITIONS FOR PREVENTION OR TREATMENT OF CHRONIC OBSTRUCTIVE PULMONARY DISEASE

(71) Applicants: INSERM (INSTITUT NATIONAL DE LA SANTE ET DE LA RECHERCHE MEDICALE), Paris (FR); UNIVERSITE PARIS-EST CRETEIL VAL DE MARNE, Creteil Cedex (FR); ASSISTANCE PUBLIQUE-HOPITAUX DE PARIS, Paris (FR); AGROPARISTECH, Paris (FR)

(72) Inventors: Jorge Bernardo Boczkowski, Creteil Cedex (FR); Serge Adnot, Creteil Cedex (FR); Maylis Dagouassat, Creteil Cedex (FR); Jean-Marie Gagliolo, Creteil Cedex (FR)

(21) Appl. No.: 14/399,352

(22) PCT Filed: May 7, 2013
(86) PCT No.: PCT/EP2013/059474

§ 371 Date: Nov. 6, 2014

(30) Foreign Application Priority Data
May 9, 2012 (EP) 12305514.7

(51) Int. Cl. A61K 31/415; G01N 33/50; A61K 31/405; A61K 31/352; A61K 31/4035

(52) U.S. Cl. CPC A61K 31/415; A61K 31/352; A61K 31/4035; A61K 31/405; G01N 33/502; G01N 2500/10
USPC 514/406; 514/455; 514/411; 514/420; 435/39; 435/18; 435/6.13; 435/6.12

(57) ABSTRACT

The present invention relates to methods and compositions for the prevention or treatment of chronic obstructive pulmonary disease.

### FIELD OF THE INVENTION

The present invention relates to methods and compositions for the prevention or treatment of chronic obstructive pulmonary disease.

### BACKGROUND OF THE INVENTION

Chronic obstructive pulmonary disease (COPD), currently the fourth leading cause of death in the United States and in Europe, will become the third leading cause by 2020 (Mannino and Buist. Lancet 2007; 370: 765-773). Cigarette smoking is the most important cause, and smoking cessation early in the course of the disease can slow the rate at which lung function is lost (Wagena et al. Respir Med 2004; 98: 805-815). An abnormal inflammatory response of the lung, which persists despite cessation of smoking (Yoshida and Tuder. Physiol Rev 2007; 87: 1047-1082), is characteristic of COPD. This inflammation is believed to play a major role in the pathogenesis and progression of COPD (Yoshida and Tuder. Physiol Rev 2007; 87: 1047-1082). However, its characteristics are not well defined.

There is a need to develop new drugs that will be suitable for preventing or treating Chronic obstructive pulmonary disease (COPD). In this way, it has been suggested that characterisation of new therapeutic targets in COPD may be highly desirable.

Cellular senescence is a state of irreversible growth arrest, which limits tissue renewal and participates in the aging process (Campisi. Cell 2005; 120: 513-522). Senescence can be linked to shorten of telomeres during continuous cell replication or be triggered by stressors, such as hydrogen peroxide or cigarette smoke (Campisi. Cell 2005; 120: 513-522) (replicative and accelerated senescence respectively). Both types of senescence may be induced through either or both the ATM/ATR-p53 and p16-retinoblastoma protein (pRb) pathways (Kim and Sharpless. Cell 2006; 127: 265-275). Importantly, senescent cells show numerous changes in gene expression, and acquire a complex phenotype that includes the secretion of many inflammatory mediators (the senescence associated secretory phenotype, SASP) than can profoundly impact cellular environment (Coppe et al. Annu Rev Pathol 2010; 5: 99-118).

Senescence of lung fibroblasts was described in patients with severe COPD (Muller et al. Respir Res 2006; 7: 32) and this process has been proposed to participate in COPD pathogenesis. However, scarce data are available concerning the mechanisms of fibroblasts senescence in COPD (Nyunoya et al. Am J Respir Crit Care Med 2009; 179: 279-287) and its relation with inflammation.

### SUMMARY OF THE INVENTION

The present invention relates to a compound which is selected from the group consisting of PGE2-receptor antagonists, PGE2-receptor expression inhibitors, COX-2 inhibitors, COX-2 expression inhibitors, prostaglandin E2 synthase inhibitors or prostaglandin E2 synthase expression inhibitors for use in the prevention or treatment of COPD in a subject in need thereof.

### DETAILED DESCRIPTION OF THE INVENTION

The role of PGE2 in COPD was investigated by inventors using COPD fibroblasts, PGE2, and PGE2-receptors EP2 and EP4 antagonists. The inventors found that COPD fibroblasts display higher levels of PGE2, PGE2 synthesis enzyme mPGE2 and PGE2-receptor EP4 than non-smoker controls and higher level of PGE2-receptor EP2 than both smoker and non-smoker controls. The inventors also demonstrated that PGE2 induce accelerated senescence and related inflammation in fibroblasts, and that the inflammatory effects of PGE₂ are secondary to senescence induction and that these effects were more important in COPD than in non-smoker and smoker controls fibroblasts. The inventors also demonstrated that PGE2 induce senescence and related inflammation in lung mice. In this way, PGE₂ is a new mediator of senescence and inflammation in COPD. Therefore, blockade of PGE₂ synthesis or receptors constitute new pharmacological approaches to overcome senescence and inflammation in COPD.

### Therapeutic Methods and Uses

Accordingly the present invention relates to a compound which is selected from the group consisting of PGE2-receptor antagonists, PGE2-receptor expression inhibitors, COX-2 inhibitors, COX-2 expression inhibitors, prostaglandin E2 synthase inhibitors or prostaglandin E2 synthase expression inhibitors for use in the prevention or treatment of COPD in a subject in need thereof.

As used herein, the term “subject” denotes a mammal. In a preferred embodiment of the invention, a subject according to the invention refers to any subject (preferably human) afflicted or at risk to be afflicted with COPD.

The method of the invention may be performed for any type of COPD such as revised in the World Health Organisation Classification of COPD and selected from the group: Chronic bronchitis (asthmatic (obstructive), emphysematous, with: airways obstruction, emphysema); Chronic obstructive (asthma, bronchitis, tracheobronchitis); Chronic obstructive pulmonary disease with acute lower respiratory infection; Chronic obstructive pulmonary disease with acute exacerbation; chronic obstructive pulmonary disease (Chronic bronchitis: asthmatic (obstructive) NOS, emphysematous NOS, obstructive NOS); Chronic obstructive pulmonary disease (Chronic obstructive: airway disease NOS, lung disease NOS).

In a particular embodiment, the compound according to the invention may be used in the prevention or treatment of senescence and inflammation in COPD in a subject in need thereof.

As used herein, the term “PGE2” has its general meaning in the art and refers to prostaglandin E2.

The term “PGE-2 receptor” has its general meaning in the art and refers to PGE-2 receptors EP1, EP2, EP3 and EP4 (Narumiya et al. (1999), Physiol. Rev. 79(4):1193-226).

The terms “COX-2” and “PTGS2” have their general meaning in the art and refer to cyclooxygenase-2 that is responsible the synthesis of PGH2, precursor of PGE-2 (Iyer et al. (2009), Expert Opin. Ther. Targets 13(7):849-865).

The term “PGES” has its general meaning in the art and refers to prostaglandin E2 synthase. The term “PGES” refers to enzymes responsible of PGE2 synthesis such as microsomal prostaglandin E2 synthase (mPGES-1 and mPGES-2) and cytosolic prostaglandin E2 synthase (cPGES).

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs which are modified, by processes such as capping, polyadenylation, methylation, and editing, and proteins (e.g., PGE-2 receptor or COX-2) modified by, for example, methylation, acetylation, phosphorylation, ubiquitination, SUMOylation, ADP-ribosylation, myristilation, and glycosylation.

An “inhibitor of expression” refers to a natural or synthetic compound that has a biological effect to inhibit the expression of a gene.

The term “PGE2-receptor antagonist” refers to a compound that selectively blocks or inactivates the PGE2-receptor. As used herein, the term “selectively blocks or inactivates” refers to a compound that preferentially binds to and blocks or inactivates PGE2-receptor with a greater affinity and potency, respectively, than its interaction with the other sub-types or isoforms of the PG-receptor family. Compounds that prefer PGE2-receptor, but that may also block or inactivate other PG-receptor sub-types, as partial or full antagonists, are contemplated. The “PGE2-receptor antagonist” may also consist in compounds that inhibit the binding of PGE2 to PGE2-receptor such as compounds having the ability to bind PGE2 with high affinity and specificity or compounds that compete with PGE2. Typically, a PGE2-receptor antagonist is a small organic molecule, a peptide, a polypeptide, an aptamer or an antibody.

In one embodiment of the invention, a PGE2-receptor antagonist is selected from EP1 antagonists, EP2 antagonists, EP3 antagonists or EP4 antagonists.

In one embodiment of the invention, PGE2-receptor antagonists include but are not limited to EP1 antagonists: ONO-8711, SC-19220, AH-6809, SC-51322, ZD-4953, ZD-6416, ZD-6840, SC-51089 described in WO03/084917; EP2 antagonists: AH6809, PF-04418948 and compounds described in U.S. patent Ser. Nos. 11/896,922, 11/896,923, 12/142,058, 12/171,365; EP3 antagonists: DG-041 (2,3-Dichlorothiophene-5-sulfonic acid, 3-[1-(2,4-dichlorobenzyl)-5-fluoro-3-methyl-1H-indol-7-yl]acryloylamide) (Heptinstall et al. (2008), Platelets, 19(8): 605-613); and EP4 antagonists: GW627368X, AH23848B, L-161982, AH22921X, EP4RA, omega-substituted prostaglandin E derivatives, 5-thia-prostaglandin E derivatives, ONO-AE3-208 (4-{4-cyano-2-[2-(4-fluoronaphthalen-1-yl)propionylamino]phenyl}butyricacid), peptides, and compounds described in U.S. patent Ser. Nos. 10/545,478, 12/752,179, International Patent Publication Nos. WO 00/15608, WO 01/42281, WO 00/18744, WO 00/03980, WO 01/10426, WO 00/21532, WO 00/18405, WO 01/72302, (Fukuda et al. (2007), Acta Obstet. Gynecol. Scand. 86(11):1297-302).

In one embodiment of the invention, a PGE2-receptor antagonist may be selected from PGE2 binding protein (U.S. patent Ser. No. 12/499,646), prostaglandin analogues or compounds described in U.S. Pat. Nos. 3,749,776, 4,004,027, 5,449,673, 5,393,747.

The PGE2-receptor antagonist of the invention may consist in an antibody or antibody fragment directed against PGE2 or PGE2-receptor.

Examples of antibodies directed against PGE2 include but are not limited to 19C9, 4F10, 15F10, K1B, K7H, K3A, L11, L21, 2B5-7.0, 2B5-8.0 or 2B5-9.0, or a variant thereof. In an embodiment, the variant is a humanized variant, such as Hu2B5.P1 or Hu2B5.P2 described in the U.S. patent Ser. No. 12/499,646.

The term “COX-2 inhibitor” refers to any compound able to prevent the action of COX-2. The COX-2 inhibitor of the present invention is a compound that inhibits or reduces the activity of COX-2.

In one embodiment, the COX-2 inhibitor of the invention is an inhibitor of COX-2 activity. Said inhibitor of COX-2 activity may be selected from the group consisting of small organic molecules, peptides, polypeptides, aptamers or antibodies (preferably intra-antibodies).

The inhibitors of COX-2 activity are well-known in the art as illustrated by Chung et al. (2005), Expert Opin. Ther. Patents 15(1):9-32.

In one embodiment of the invention, the inhibitor of COX-2 activity is selected from the group consisting of nimesulide, 4-hydroxynimesulide, flosulide, meloxicam, L 475 L337, Vioxx, SC 58125, Celecoxib, NS 398, DuP 697, Indomethacin, Valdecoxib, Meloxicam, Rofecoxib, Etoricoxib, sulindac, Lumiracoxib, E-522, NS-398 and compounds disclosed in U.S. patent Ser. No. 10/545,478, and International Patent Publication Nos. WO 04/72037, WO 04/72057.

The term “prostaglandin E2 synthase inhibitor” refers to any compound able to prevent the action of prostaglandin E2 synthase. The prostaglandin E2 synthase inhibitor of the present invention is a compound that inhibits or reduces the activity of prostaglandin E2 synthase.

In one embodiment, the prostaglandin E2 synthase inhibitor of the invention is an inhibitor of prostaglandin E2 synthase activity. Said inhibitor of prostaglandin E2 synthase activity may be selected from the group consisting of small organic molecules, peptides, polypeptides, aptamers or antibodies (preferably intra-antibodies).

The inhibitors of prostaglandin E2 synthase activity are well-known in the art as illustrated by Iyer et al. (2009), Expert Opin. Ther. Targets 13(7):849-865.

In one embodiment of the invention, the inhibitor of prostaglandin E2 synthase activity is selected from the group consisting of fatty acids and prostaglandin analogues, 15-deoxy-PGJ2, LTC4, U-51605, NS-398, Sulindac, MK-886, MF-63, Thienopyrroles, Benzoxazole, Naphthalene disulfonamide, 3-benzamidocarbazole and compounds disclosed in Iyer et al. (2009), Expert Opin. Ther. Targets 13(7):849-865.

In another embodiment, the PGE2-receptor antagonist, COX-2 inhibitor or PGE2 synthase inhibitor of the invention is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against the compound of the invention as above described, the skilled man in the art can easily select those inhibiting PGE2-receptor, COX-2 or PGE2 synthase.

In one embodiment, the compound of the invention is an inhibitor of PGE2-receptor expression, inhibitor of COX-2 expression or inhibitor of prostaglandin E2 synthase expression.

Inhibitors of PGE2-receptor expression, inhibitors of COX-2 expression or inhibitors of prostaglandin E2 synthase expression for use in the present invention may be based on antisense oligonucleotide constructs. Anti-sense oligonucleotides, including anti-sense RNA molecules and anti-sense DNA molecules, would act to directly block the translation of PGE2-receptor, COX-2, or prostaglandin E2 synthase mRNA by binding thereto and thus preventing protein translation or increasing mRNA degradation, thus decreasing the level of PGE2-receptor, COX-2 or prostaglandin E2 synthase proteins, and thus activity, in a cell. For example, antisense oligonucleotides of at least about 15 bases and complementary to unique regions of the mRNA transcript sequence encoding PGE2-receptor, COX-2 or prostaglandin E2 synthase can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of PGE2-receptor, COX-2 or prostaglandin E2 synthase expression for use in the present invention. PGE2-receptor, COX-2 or prostaglandin E2 synthase gene expression can be reduced by contacting the subject or cell with a small double stranded RNA (dsRNA), or a vector or construct causing the production of a small double stranded RNA, such that PGE2-receptor, COX-2 or prostaglandin E2 synthase expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of PGE2-receptor, COX-2 or prostaglandin E2 synthase expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of PGE2-receptor, COX-2 or prostaglandin E2 synthase mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of PGE2-receptor, COX-2 or prostaglandin E2 synthase expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing PGE2-receptor, COX-2 or prostaglandin E2 synthase. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Chiffon, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the compounds of the invention may be used concomitantly or sequentially in the prevention or treatment of COPD in a subject in need thereof.

In another particular embodiment, the compounds of the invention may be used concomitantly or sequentially in the prevention or treatment of senescence and inflammation in COPD in a subject in need thereof.

In one embodiment, the present invention relates to a method for preventing or treating COPD in a subject in need thereof, comprising the step of administering to said subject a compound which is selected from the group consisting of PGE2-receptor antagonists, PGE2-receptor expression inhibitors, COX-2 inhibitors, COX-2 expression inhibitors, prostaglandin E2 synthase inhibitors or prostaglandin E2 synthase expression inhibitors.

In a particular embodiment, the method according to the invention may be used in the prevention or treatment of senescence and inflammation in COPD in a subject in need thereof.

### Pharmaceutical Composition

The compound of the invention may be used or prepared in a pharmaceutical composition.

In one embodiment, the invention relates to a pharmaceutical composition comprising the compound of the invention and a pharmaceutical acceptable carrier for use in the prevention or treatment of COPD in a subject of need thereof.

Typically, the compound of the invention may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The compound of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

### Screening method

In a further aspect, the present invention relates to a method of screening a candidate compound for use as a drug for the prevention or treatment of COPD in a subject in need thereof, wherein the method comprises the steps of: i) providing candidate compounds and ii) selecting candidate compounds that blocks PGE2-receptor, inhibit PGE2-receptor expression, inhibit COX-2 activity, inhibit COX-2 expression, inhibit prostaglandin E2 synthase activity or inhibit prostaglandin E2 synthase expression.

In a further aspect, the present invention relates to a method of screening a candidate compound for use as a drug for the prevention or treatment of COPD in a subject in need thereof, wherein the method comprises the steps of:
providing a PGE2, PGE2-receptor, COX-2, prostaglandin E2 synthase, providing a cell, tissue sample or organism expressing the PGE2-receptor,
providing a candidate compound such as small organic molecule, prostaglandin analogues, antibodies, peptide or polypeptide,
measuring the activity of the PGE2-receptor, COX-2 or prostaglandin E2 synthase,
and selecting positively candidate compounds that blocks PGE2-receptor, inhibit PGE2-receptor expression, inhibit COX-2 activity, inhibit COX-2 expression, inhibit prostaglandin E2 synthase activity or inhibit prostaglandin E2 synthase expression.

Methods for measuring the activity of the PGE2-receptor, COX-2 or prostaglandin E2 synthase are well known in the art. For example, measuring the PGE2-receptor activity involves determining a Ki on the PGE2-receptor cloned and transfected in a stable manner into a CHO cell line or measuring one or more of the second messengers of the PGE2-receptor (cAMP, PI3K, AKT, PPARγ) in the presence or absence of the candidate compound.

Tests and assays for screening and determining whether a candidate compound is a PGE2-receptor antagonist are well known in the art (U.S. Pat. Nos. 5,393,747; 4,004,027). In vitro and in vivo assays may be used to assess the potency and selectivity of the candidate compounds to reduce PGE2-receptor activity.

Activities of the candidate compounds, their ability to bind PGE2-receptor and their ability to inhibit PGE2-receptor activity may be tested using isolated fibroblats expressing PGE2-receptor, CHO cell line cloned and transfected in a stable manner by the human PGE2-receptor.

Cells and fibroblast expressing another receptor than PGE2-receptor may be used to assess selectivity of the candidate compounds.

The invention will be further illustrated by the following figures and examples. However, these examples and figures should not be interpreted in any way as limiting the scope of the present invention.

### FIGURES

FIG. 1: Expression of senescent markers in fibroblasts.

(A) Replicative senescence of pulmonary fibroblasts derived from patients with chronic obstructive pulmonary disease (COPD, n=25) and non-smoker and smoker controls (NS-C, n=10; S-C, n=25 respectively). The rate of proliferation was evaluated by the sum of population doubling level (PDL). * p<0.05 COPD versus controls. (B) Percentage of Senescence associated (SA) β-Gal-positive cells. *p<0.05 COPD versus controls. (C) Telomere length. The T/S ratio is the ratio of telomere repeated copy number over single-gene copy number (36B4 gene). * p<0.05 passage 3 vs. passage 7. (D) Quantitative transcriptional expression of p16 genes by real time qPCR. ** p<0.01 COPD versus NS-C; † p<0.05 COPD vs. S-C; §p<0.05 and §§§p<0.001 passage 3 vs. passage 7. Data are presented as mean±SEM in the whole Figure.

FIG. 2: Up-regulation of the PGE₂/EP₂/EP₄ pathway in COPD fibroblasts at non-senescent and senescent stages.

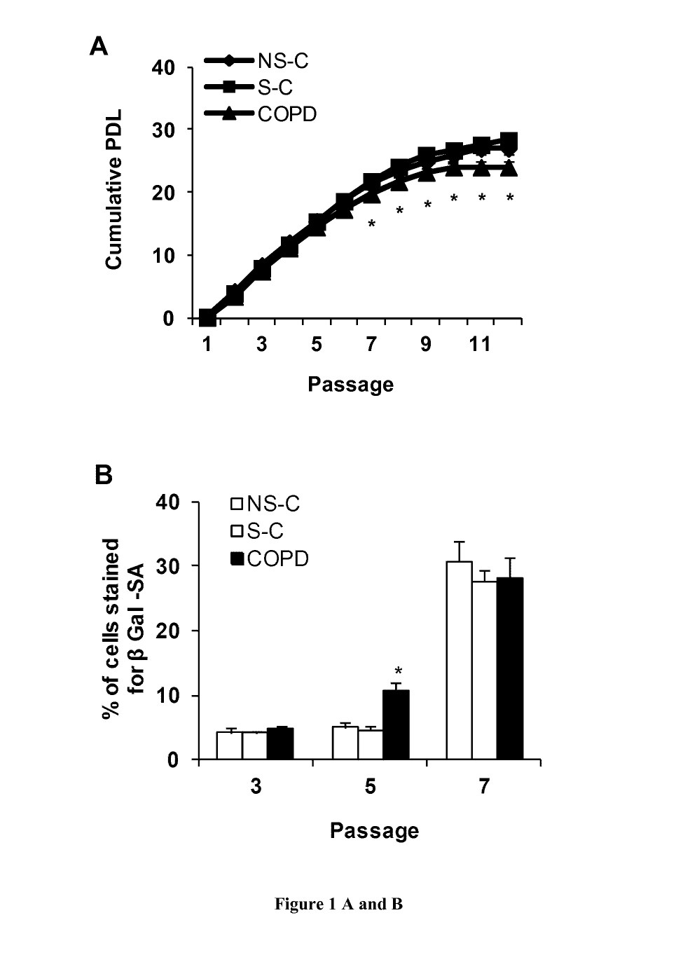

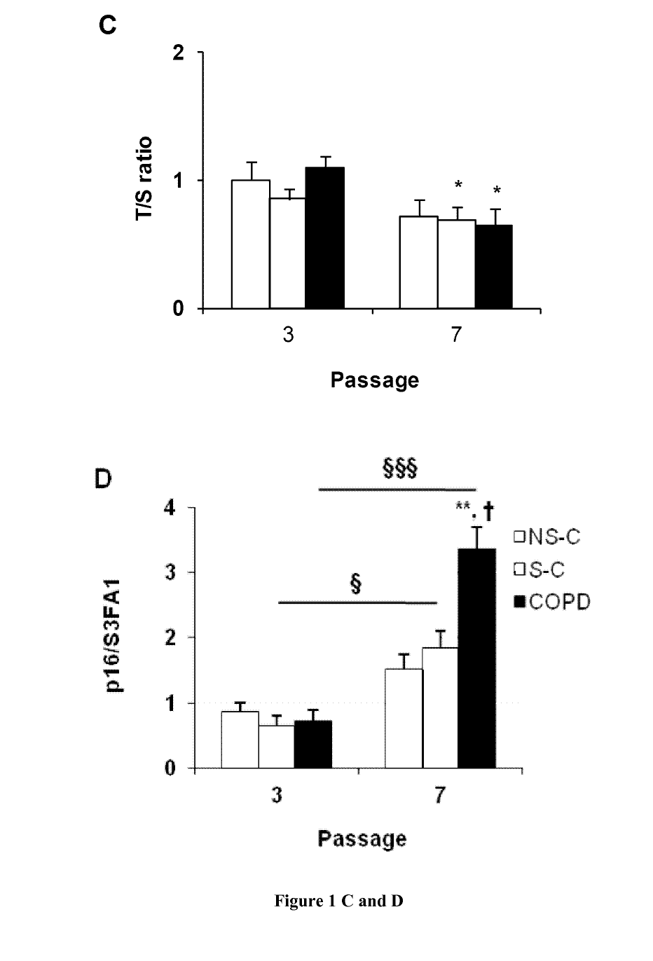

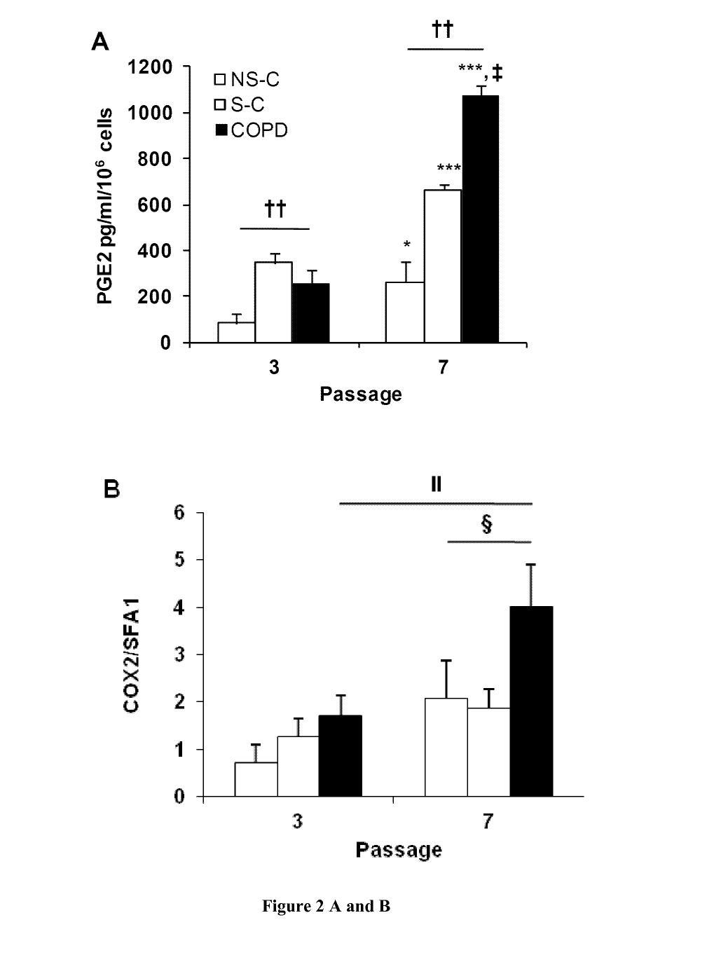

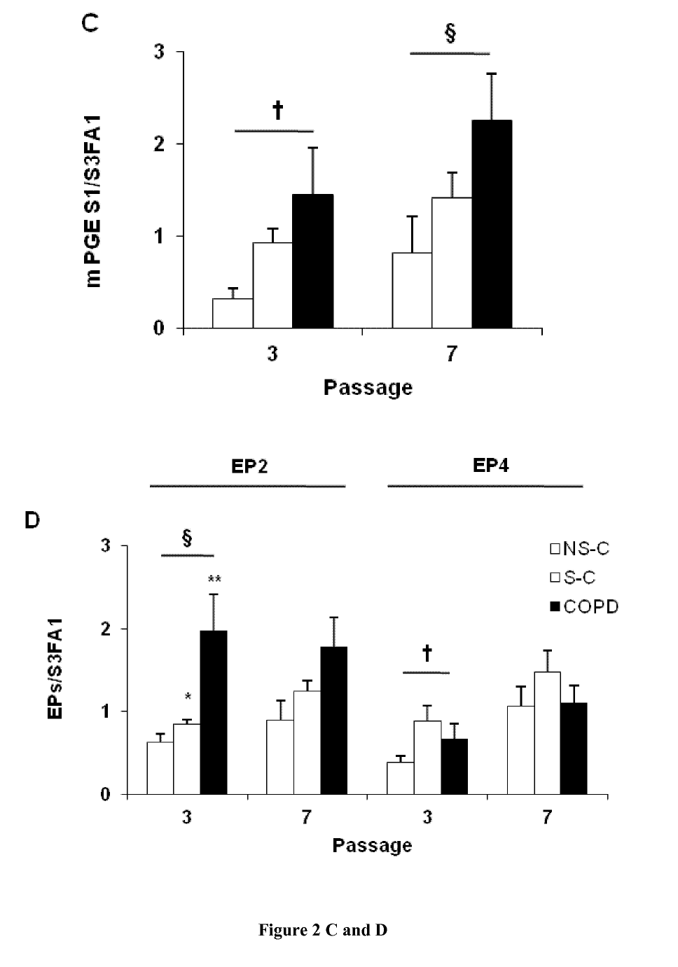

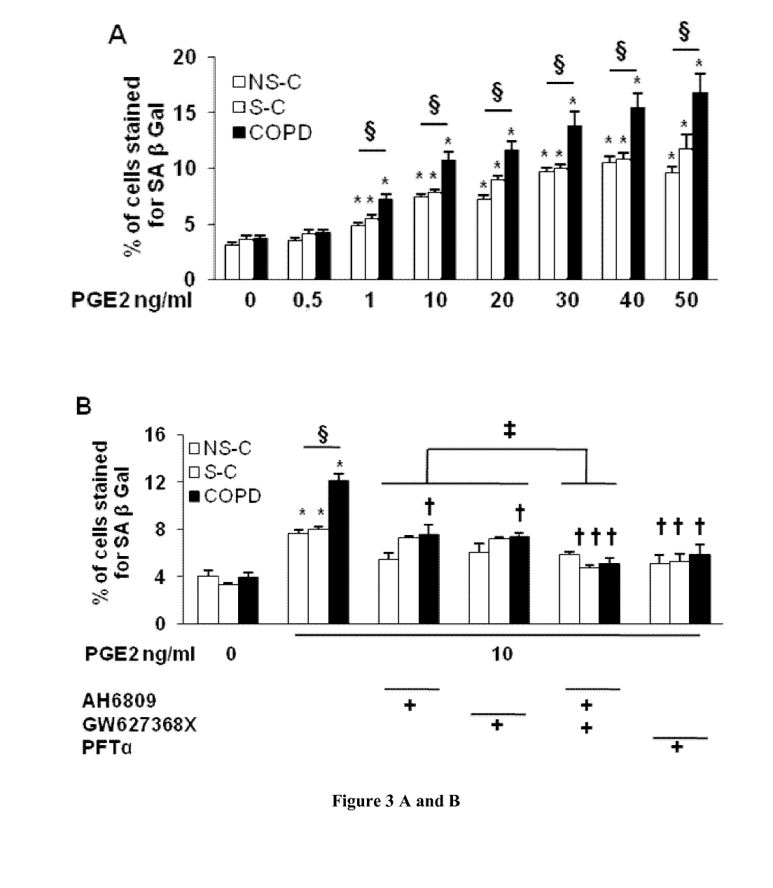

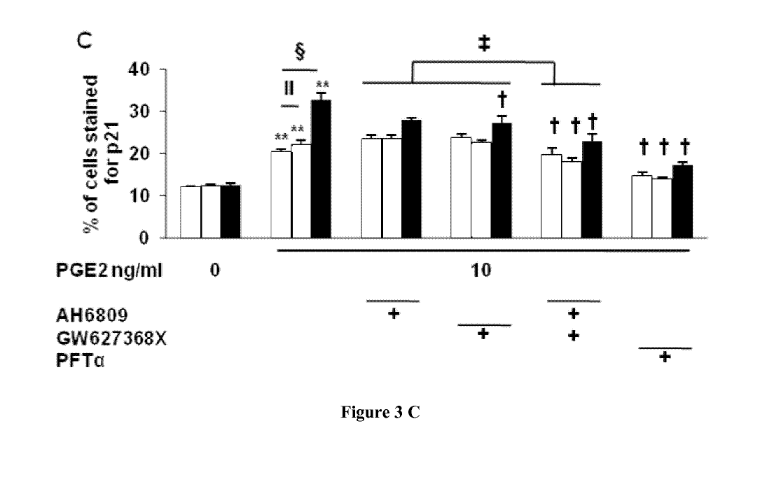

Abbreviations are the same as those described in FIG. 1. (A) PGE₂ levels in culture media, n=6, 14 and 16 for NS-C, S-C, and COPD respectively, *** p<0.001 passage 7 vs. passage 3; †† p<0.01 S-C and COPD vs. NS-C. Quantitative transcriptional expression of COX2 (B), mPGE₂ (C), EP₂, EP₄ (D) genes by real time qPCR. * p<0.05, ** p<0.01 S-C or COPD vs NS-C; † p<0.05 S-C and COPD vs. NS-C; §p<0.05, COPD vs. controls; ll p<0.05 passage 3 vs. passage 7. Data are presented as mean±SEM in the whole Figure.

FIG. 3: PGE₂ induces the senescence of non-senescent fibroblasts in dose-dependent manner.

Abbreviations are the same as those described in FIG. 1. (A) Percentage of SA β-Gal-positive cells. NS-C, S-C and COPD fibroblasts (n=5, 6, and 6 respectively) were exposed to different concentrations of PGE₂ and stained after 24 hours exposure. * p<0.05 PGE₂ vs. vehicle (DMSO). §p<0.05 COPD vs. controls (B) Bar graph of cells stained for SA β-Gal activity after pre-treatment with antagonists of PGE₂ receptors [EP₂ (AH6809 (10 μM), EP₄ (GW627368X (10 μM)] or PFT-α and exposure to PGE₂ at 10 ng·ml⁻¹. n=6. *p<0.05 PGE₂ vs. DMSO; † p<0.05 PGE₂ vs. PGE₂+PFT-α or PGE₂+ antagonists; ‡ p<0.05 both antagonists vs. one antagonist. (C) Bar graph of cells stained for p21 after the same treatments as the ones described in section (D). ** p<0.01 PGE₂ vs. DMSO; ll p<0.05 S-C vs. NS-C; §p<0.05 COPD vs. controls; † p<0.05 PGE₂ vs. PGE₂+PFT-α or PGE₂+ antagonists; ‡ p<0.05 both antagonists vs. one antagonist. Data are presented as mean±SEM in the whole Figure.

### EXAMPLE

### The COX2/PGE2 Pathway Maintains Senescence of Chronic Obstructive Pulmonary Disease Fibroblasts

### Methods

### In Vitro Experiments

Patients and Cells:

Primary lung fibroblasts were isolated by the explant technique (Normand J and Karasek M A. In Vitro Cell Dev Biol Anim 1995; 31:447-455) from lung specimens obtained for lung tumor resection from 25 patients with COPD and 35 subjects without clinical, morphological or functional signs of COPD (controls) (Table 1). Controls were divided into non-smoker and smoker groups (NS-C, n=10 and S-C, n=25 respectively). Smoking status (current smokers or ex-smokers, defined by smoking cessation after more than 1 year (Lapperre T S et al., Respir Res 2007; 8:85; Savale L et al. Am J Respir Crit Care Med 2009), was considered in the analysis of the different results (Table 2). Classification of COPD severity was based on the 2003 Global initiative for chronic obstructive lung disease (GOLD) criteria (Rabe K F et al., Am J Respir Crit Care Med 2007; 176:532-555). Informed consent was obtained from all patients and the study was approved by the “Comite de Protection des Personnes Ile de France IX”.

TABLE 1
Patient characteristics.

| | Non smokers | Smokers | COPD | p |
|---|---|---|---|---|
| | (NS-C) | (S-C) | smokers | value |
| Patients (n) | 10 | 25 | 25 | |
| Age (yrs) | 64 (42-80) | 62 (51-77) | 67 (51-80) | ns |
| Sex (M/F) | 4/6 | 13/12 | 19/6 | |
| FEV₁/VC | 80 (75-86) | 79 (73/107) | ††† 59 (53/69) | p < 0.001 |
| FEV₁ (%) | 102 (87-119) | 89 (76-113) | ***/‡‡ 73 (50-113) | p < 0.001 |
| GOLD (n) 0/I/II/III/IV | 10/0/0/0/0 | 25/0/0/0/0 | 0/9/16/0/0 | |
| Smoking history (pack-yrs) | 0 | 46 (20-100) | 50 (20-100) | ns |
| Emphysema score(%) | 3.5 ± 1.9 | 12.5 ± 3.5 | †† 42.6 ± 6.5 | p < 0.001 |

Abbreviations: COPD, chronic obstructive pulmonary disease; VC, vital capacitiy; FEV₁, forced expiratory volume in 1 sec; GOLD, Global Initiative for Chronic Obstructive Lung Disease.
Emphysema score was assessed on 0 to 100% scale.
Data are expressed as median with minimum and maximum quartiles (in parentheses), except for emphysema score, which is expressed as mean ± SEM.
†† p < 0.01,
††† p < 0.001 COPD patients versus controls (NS-C and S-C);
*** p < 0.001 COPD patients versus non-smokers;
‡‡ p < 0.01 COPD patients versus smokers.

Abbreviations: COPD, chronic obstructive pulmonary disease; VC, vital capacity; FEV₁, forced expiratory volume in 1 sec; GOLD, Global Initiative for Chronic Obstructive Lung Disease. Emphysema score was assessed on 0 to 100% scale.

Data are expressed as median with minimum and maximum quartiles (in parentheses), except for emphysema score, which is expressed as mean±SEM. †† p<0.01, ††† p<0.001 COPD patients versus controls (NS-C and S-C); *** p<0.001 COPD patients versus non-smokers; ‡‡ p<0.01 COPD patients versus smokers.

TABLE 2
Clinical and biological data of fibroblasts from non-smoker, current and ex-smoker controls and COPD patients.

| | Non | Current | Ex | COPD | COPD |
|---|---|---|---|---|---|
| | smokers | smokers | Smokers | smokers | ex-smokers |
| | (NS-C) | (S-C) | (ExS-C) | (S-COPD) | (ExS-COPD) |
| Patients (n) | 10 | 12 | 13 | 15 | 10 |
| Age (years) | 64 (42-80) | 65 (43-77) | 60 (51-79) | 68 (59-80) | 61 (51-74) |
| Smoking history (PA) | 0 | 43 (12-100) | 45 (23-60) | 53 (18-80) | 45 (20-100) |
| Arrest (years) | — | — | 13 (5-26) | — | 14 (4-27) |
| FEV1/VC | 80.1 (75-86) | 77.2 (74-105) | 83.3 (74-98) | 62.4 (67-54) ** | 64 (66-53) *** |
| Cumulative PDL at passage 7 (P7) | 23.2 ± 0.6 | 23.3 ± 0.9 | 22.9 ± 1.1 | 19.2 ± 0.8 * ‡ | 22.3 ± 0.7 |
| S A β-gal activity at passage 5 (%) | 6.2 ± 0.8 | 5.1 ± 0.2 | 4.7 ± 0.7 | 13.86 ± 0.9 * | 11.8 ± 0.75 * |
| p16/S3FA1 at P3 | 0.6 ± 0.2 | 0.4 ± 0.1 | 0.5 ± 0.2 | 0.6 ± 0.12 | 0.5 ± 0.04 |
| p16/S3FAl at P7 | 2.2 ± 0.2 £ | 2.1 ± 0.4 § | 1.7 ± 0.25 § | 3.1 ± 0.7 * §§ | 3.5 ± 0.6 * §§ |
| p53/S3FA1 at P3 | 1.1 ± 0.1 | 0.8 ± 0.11 | 0.9 ± 0.1 | 0.7 ± 0.2 | 0.6 ± 0.1 |
| p53/S3FA1 at P7 | 0.6 ± 0.1 | 0.8 ± 0.1 | 0.8 ± 0.1 | 0.95 ± 0.1 § | 1.1 ± 0.1 § |
| p21/S3FA1 at P3 | 0.6 ± 0.1 | 0.5 ± 0.02 | 0.7 ± 0.1 | 0.6 ± 0.1 | 0.5 ± 0.1 |
| p21/S3FA1 at P7 | 1 ± 0.4 | 1.1 ± 0.3 § | 1.5 ± 0.1 §§ | 1.3 ± 0.2 §§ | 1.9 ± 0.2 §§ |
| IL6 pg/ml at P3 | 2107 ± 275 | 1907 ± 331 | 2039 ± 554 | 2040 ± 454 | 2351 ± 752 |
| IL6 pg/ml at P7 | 3230 ± 525 | 3440 ± 685 § | 3930 ± 520 § | 5980 ± 901 * § | 6150 ± 1220 * § |
| IL8 pg/ml at P3 | 3434 ± 815 | 3158 ± 735 | 2887 ± 447 | 2421 ± 565 | 3801 ± 1500 |
| IL8 pg/ml at P7 | 4088 ± 1870 | 4850 ± 530 | 5160 ± 1060 | 10200 ± 2020 * § | 8460 ± 2023 * § |
| CX3CL1 pg/ml at P3 | 227 ± 54 | 330 ± 12.6 | 271 ± 34 | 242 ± 43 | 332 ± 45 |
| CX3CL1 pg/ml at P7 | 339 ± 105 | 486 ± 100 | 478 ± 90 £ | 778 ± 175 * §§ | 883 ± 203 * §§ |
| PGE₂ pg/ml at P3 | 83.1 ± 40.5 | 369.9 ± 71.5 | 327.2 ± 44.1 | 358.9 ± 66.4 | 215.1 ± 58.9 |
| PGE₂ pg/ml at P7 | 264.9 ± 38 § | 719.3 ± 20.6 § | 609.0 ± 81.3 § | 1109.6 ± 149.6 * §§ | 1029.6 ± 220.4 * §§ |
| EP₂/S3FA1 at P3 | 0.6 ± 0.1 | 0.9 ± 0.1 † | 0.7 ± 0.1 | 2.1 ± 0.5 * | 1.8 ± 0.2 * |
| EP₂/S3FA1 at P7 | 0.9 ± 0.2 | 1 ± 0.2 | 1.6 ± 0.1 § | 1.7 ± 0.4 | 1.8 ± 0.4 |
| EP₄/S3FA1 at P3 | 0.4 ± 0.1 | 1.2 ± 0.3 † | 0.6 ± 0.1 | 0.8 ± 0.2 † | 0.8 ± 0.3 † |
| EP₄/ S3FA1 at P7 | 1.3 ± 0.2 | 1.4 ± 0.4 | 1.5 ± 0.3 £ | 1.03 ± 0.23 | 1.15 ± 0.24 |

Abbreviations COPD, chronic obstructive pulmonary disease; VC, vital capacity; FEV₁, forced expiratory volume in 1 sec.
Age, smoking history, smoking arrest and FEV1/VC are expressed as median with minimum and maximum quartiles (in parentheses); the other data are expressed as ±SEM.
* p < 0.05,
** p < 0.01,
*** p < 0.001 COPD patients versus controls (NS-C, S-C, ExS-C);
§ p < 0.05,
§§ p < 0.01 Passage 7 (P7) versus Passage 3 (P3);
† p < 0.05 COPD patients or S-C versus NS-C;
‡ p < 0.05 S-COPD versus ExS-COPD.

Abbreviations COPD, chronic obstructive pulmonary disease; VC, vital capacity; FEV₁, forced expiratory volume in 1 sec. Age, smoking history, smoking arrest and FEV1/VC are expressed as median with minimum and maximum quartiles (in parentheses); the other data are expressed as mean±SEM. * p<0.05, ** p<0.01, *** p<0.001 COPD patients versus controls (NS-C, S-C, ExS-C); §p<0.05, §§p<0.01 Passage 7 (P7) versus Passage 3 (P3); † p<0.05 COPD patients or S-C versus NS-C; ‡ p<0.05 S-COPD versus ExS-COPD.

Cells Treatments:

Fibroblasts were exposed to either PGE₂ (0.5 to 50 ng ml⁻¹) or conditioned medium (CM) collected from fibroblasts incubated for 24 h. In different experiments cells were treated with inhibitors of COXs, agonists or antagonists of prostaglandin receptors EP₂ and EP₄, the antioxidant N-acetylcysteine (NAC), or the inhibitor of p53 transcriptional effects pifithriñ-\tilde{α}.

Characterization of Fibroblasts Senescence:

Senescence was characterized by measuring cumulative population doubling levels (PDLs) (Holz O et al., Eur Respir J 2004; 24:575-579), telomere length (Savale L et al., Am J Respir Crit Care Med 2009), senescence associated β-galactosidase activity (SA β-Gal) (Dimri G P. Cancer Cell 2005; 7:505-512), and the expression of phospho (serine 15)-p53 p16, p21, and caspase 3 by western blot, immunofluorecence and RT-qPCR analysis. The level of 29 soluble factors in conditioned medium was quantified using the Luminex technology (Millipore, Molscheim, France).

Analysis of the PGE₂ Pathway:

PGE₂ was quantified by using the Prostaglandin E₂ monoclonal EIA kit (Bertin Pharma, Montigny le Bretonneux, France). PGE₂ receptors and enzymes involved in PGE₂ synthesis were quantified by RT-qPCR. Cellular reactive oxygen species production was quantified by the DCFH-DA assay (Carter W et al., J Leukoc Biol 1994; 55:253-258).

Animal Experiments

Male 8-week-old mice of C57BL/6 background and p53−/− animals (kindly provided by Tyler Jacks, MIT, Cambridge) were intratracheally instilled with either PGE₂ (1 ng·ml⁻¹) or vehicle (DMSO 1/10000). Instilled volume was 50 μl. Twenty-four and 48 h after instillation mice were sacrificed and analysis of lung mRNA expression of senescence markers and cytokines were performed. Studies were conducted in compliance with INSERM guidelines regarding the fair treatment of animals.

Statistical Analysis

Data were analyzed with GraphPad Prism 4.0 (La Jolla, Calif., USA). Comparisons between groups were performed with Kruskall-Wallis' non-parametric analysis of variance test followed by two-by-two comparisons with Mann-Whitney's U test when a significant difference was detected. Correlations were analyzed using the Spearman's rank correlation coefficient. p<0.05 was considered statistically significant.

Results

Patient's Clinical and Demographic Features

The clinical and demographic features of the subjects are presented in Tables 1 and 2. The three groups were similar in age. Smokers and COPD patients had similar smoking history. Subjects with COPD displayed a mild to moderate degree of disease as revealed by GOLD stages I and II (9 and 16 subjects respectively) and an emphysema score of 42.6±6.5 over 100 (Bankier A A et al., Radiology 1999; 211:851-858).

Fibroblasts from COPD Patients Displayed a Higher Replicative Senescence Compared to Controls

Fibroblasts of the three groups of patients did not express markers of cancer-associated fibroblast. Fibroblasts from COPD patients were not different from controls at non-senescent passages, but displayed a lower PDL (FIG. 1A), an increased SA β-gal staining (FIG. 1B), an increased p16 mRNA and protein expression (both in vitro and in situ in lung slides, FIG. 1D), HO-1 mRNA expression and ROS production as compared to controls at passages 5-7. In addition COPD fibroblasts showed an increased p53 and p21 protein and mRNA expression and a reduced telomere length (FIG. 1C) at passage 7 as compared to passage 3, these last two results being also observed in S-C. Cumulative PDL in COPD patients correlated positively with FEV₁/VC and FEV₁ (r=0.42 p<0.05 and r=0.40, p<0.05 respectively). It has to be noted that the difference between COPD and controls in terms of SA β-gal staining occurred at passage 5 (FIG. 1B) whereas that concerning p16 and cumulative PDL occurred at passage 7 (FIGS. 1A and D). Such dissociation was reported previously (Zdanov S et al., Exp Cell Res 2007; 313:3046-3056; Noureddine H et al. Circ Res; 109:543-553) and could be related to different mechanisms underlining the different markers of senescence (Kurz D J et al. J Cell Sci 2000; 113 (Pt 20):3613-3622).

Given these results, further studies were performed at passages 3, 5 and 7, considered as non-senescent, intermediate and senescent passages respectively. Analysis of the fibroblasts secretome showed that senescent COPD fibroblasts secreted higher amounts of IL-6, IL-8, GRO, CX3CL1 (Fractalkin), TNF-α, IGFBP-7, FGF-2, and CCL5 (Rantes) as compared to controls.

Smoking cessation did not affect senescence and inflammatory secretome of the 2 groups at both passages 3 and 7, except for cumulative PDL at passage 7 in COPD fibroblasts, which was significantly higher in ex-smokers than in current S-C fibroblasts (Table 2, p<0.05).

These results show that fibroblasts from mild to moderate COPD patients display a greater senescent phenotype associated with a SASP than controls.

Up-Regulation of the PGE₂ Pathway in COPD Fibroblasts

The inventors then analyzed the PGE₂ pathway in COPD and control fibroblasts. Secreted PGE₂ levels and mRNA expression of mPGES-1, the inducible isoform of the terminal enzyme in PGE₂ synthesis, were significantly higher in S-C and COPD as compared to NS-C groups at passage 3 (FIGS. 2A and C). At passage 7, PGE₂ levels were significantly higher in each group as compared to passage 3, and in COPD versus control groups (FIG. 2A). In addition, at passage 7 COX2 mRNA levels were significantly higher in COPD versus controls (FIG. 2B). This difference results probably from a high value at passage 3 (although not statistically different from controls) and similar fold increase from passage 3 as compared to controls.

PGE₂ acts through specific receptors, named EP₁₋₄ (Woodward D F et al., Pharmacol Rev 2011; 63:471-538). At passage 3, mRNA expression of EP₂ and EP₄ was significantly higher in COPD fibroblasts as compared to control groups (FIG. 2D), whereas no difference was observed for EP₁ and EP₃ (Figure E7 in the online data supplement). No significant difference in the expression of the four PGE₂ receptors was observed at passage 7 (FIG. 2D).

Overall, these results indicate an up-regulation of the PGE₂/EP₂-EP₄ pathway in COPD versus control fibroblasts.

A Single Exposure to PGE₂ Induces Accelerated Senescence and Related Inflammation of Lung Fibroblasts

The inventors next questioned whether a single exposure to PGE₂ at concentrations close to those found in the secretome of senescent fibroblasts could induce senescence of non-senescent fibroblasts, and whether COPD fibroblasts were more susceptible to this effect than controls cells.

Twenty-four hours incubation of non-senescent fibroblasts with PGE₂ induced a dose-response increase in the percentage of cells expressing SA β-gal, p21 and p16 protein (FIG. 3A) along with an increase in the levels of IL-6, CX3CL1, VEGF, FGF2, MMP-2 and TIMP-2 in cell culture supernatant. These inductions were significantly higher in COPD as compared to control fibroblasts. An absence of cleaved caspase-3 and no detection of phosphatidyl serine residues were observed in fibroblasts of the three groups of patients.

Blockade of EP₂ or EP₄ receptors with AH6809 and GW627368X respectively significantly reduced PGE₂-induced SA \tilde{β}-gal and p21 expression in COPD fibroblasts, with a small but significant higher effect when both antagonists were used simultaneously (FIGS. 3B and C). In both control fibroblasts, only the simultaneous application of both antagonists decreased PGE₂-induced SA-β gal and p21 expression (FIGS. 3B and C). Accordingly, both antagonists were needed to suppress the increase in IL-6, CX3CL1, FGF2, VEGF, MMP2 and TIMP2 induced by PGE₂. Similar effects were observed with other EP₂ and EP₄ receptor antagonists (PF-04418948 and L-161982 respectively) in PGE₂-induced SA β-gal and p21 expression in COPD and control fibroblasts. Finally, EP₂ and EP₄ agonists ONO-AE1-259-01 and CAY10598 respectively, mimicked PGE₂ effects on SA \tilde{β}-gal and p21 expression in COPD and control fibroblasts, with a small but significant higher effect when both agonists were used simultaneously, thus confirm the involvement of these receptors in PGE₂-induced senescence.

Finally, since p21 is a main downstream effector of p53 (Zhang H. J Cell Physiol 2007; 210:567-574), the inventors investigated the role of p53 on PGE₂-induced SA-β gal, p21 expression and inflammatory cytokines secretion. Pifithrin-α, an inhibitor of nuclear p53 translocation (Komarov P G et al., Science 1999; 285:1733-1737), completely prevented SA β-gal, p21 expression (FIGS. 3B and C) and the increase in IL6, CX3CL1, MMP-2 and TIMP-2 levels induced by PGE₂ in both control and COPD fibroblasts, without modification of the increase in p16.

The role of p53 in PGE₂ induced senescence and inflammation was further confirmed in wild type and p53−/− mice. A single intra-tracheal instillation of PGE₂ induced the pulmonary expression of p53 and p21 at 24 h and 48 h respectively, as well as the expression of the same inflammatory mediators induced in vitro (IL-6, CX3CL1, FGF2, VEGF, MMP2), with no change in p16 expression. In addition PGE₂ increased COX2 mRNA levels at 48 h. The induction of p21 and inflammatory mediators by PGE₂ were not observed in p53−/− mice, supporting a role of p53 in these phenomena.

Since, ROS are involved in the induction of senescence (Campisi J. Cell 2005; 120:513-522), the inventors investigated their involvement in the effects of PGE₂. The concentration of intracellular ROS and the expression of the oxidant-sensitive protein HO-1 increased in a dose-dependent manner only in fibroblasts from COPD patients incubated with PGE₂. No activation of the DNA damage-activated transcription factor ATM was observed in PGE₂-exposed COPD cells. Twenty-four hours pre-treatment of fibroblasts with both antagonists of EP₂/EP₄ receptors, the thiol antioxidant N-acetyl-cysteine (NAC), the COX1 and 2 inhibitor indomethacin, or the COX2 inhibitor celecoxib, significantly reduced the increase in ROS, and SA β-gal and p21 expression induced by PGE₂. Furthermore, exogenous PGE₂ induced synthesis of endogenous PGE₂, which was abolished by celecoxib treatment.

Collectively, these results indicate that a single exposure to PGE₂ induces senescence and associated inflammation via an EP₂/EP₄, COX2-dependent ROS and p53 cascade, and that COPD fibroblasts are significantly more susceptible to these effects than controls cells.

PGE₂ is Responsible for the Paracrine Senescent Effect of Secretome from Senescent COPD Fibroblasts

The inventors next analyzed if PGE₂ was involved in a paracrine pro-senescent effect of secretome from senescent COPD fibroblasts. Since NS-C and S-C fibroblasts behave similarly in terms of EP receptors expression, the inventors used only S-C fibroblasts as controls in these experiments.

Non-senescent fibroblasts from S-C and COPD patients (further called target fibroblasts) were incubated 24 h with conditioned medium (CM) from cells of the respective groups obtained at non-senescent and senescent passages. Incubation of target fibroblasts with CM obtained at non-senescent passage demonstrated an increase in the percentage SA-β gal positive cells only in the case of COPD. Both S-C and COPD CM obtained at senescent passage increased the percentage SA-β gal, phospho (serine 15)-p53, p21, and p16 protein expression, as well as IL-6, IL-8, MCP-1 and FGF-2 mRNA expression in target cells. However, except for MCP-1, these increases were significantly higher after incubation with COPD than with S-C CM. Senescent CM did not induced cleaved caspase-3 expression.

Treatment of fibroblasts with COX inhibitors indomethacin or celecoxib reduced by 60% the levels of PGE₂ in CM. Incubation of target cells with these media resulted in a parallel significant reduction in the percentage of SA β-gal positive cells and p21 protein expression.

Pre-treatment of target fibroblasts with pifithrin-α or both EP₂/EP₄ antagonists suppressed the increase in SA β-gal positive cells and the induction of p21 protein by S-C and COPD senescent CM. Pifithrin-α, suppressed the increase in IL-6, IL-8 and MCP-1 mRNA expression induced by CM, whereas the increase in FGF-2 was not modified.

Overall these results indicate that PGE₂ acts in a paracrin fashion to induce senescence with inflammation in COPD fibroblasts via an EP₂/EP₄-p53 cascade.

PGE₂ Enhances Replicative Senescence and Related Inflammation of Lung Fibroblasts

After demonstrating that a single exposure to PGE₂ induced senescence in human fibroblasts and mice, the inventors analyzed the effects of repetitive exposures to PGE₂ on replicative senescence. These experiments were performed only in S-C fibroblasts, since both NS-C and S-C fibroblasts behave similarly in terms of senescence and response to PGE₂.

Repeated exposures of S-C and COPD fibroblasts to PGE₂ at 1 ng·ml⁻¹ during culture from passage 3 to 9 further reduced senescence-associated cumulative PDL, and enhanced induction of SA-β gal, p21, p16, COX2 and HO-1 expression, and levels of IL-6, IL-8, GRO, VEGF and CX3CL, whereas expression of MCP3 was not modified. These phenomena were greater in COPD than in S-C fibroblasts. PGE₂ did not modify the reduction in telomeres length, but enhanced the expression of phosphorylated-ATM in COPD fibroblasts, suggesting the occurrence of DNA damage after repeated exposure to PGE₂.

Finally, the inventors investigated if endogenous PGE₂ was involved in replicative senescence. The inventors first incubated COPD and S-C fibroblasts during successive passages with indomethacin and celecoxib. Each inhibitor prevented the increase in SA-β gal, p21, p16, and cumulative PDL in COPD cells, whereas they did not modify their levels in S-C cells. This effect was followed by a parallel decrease in IL-6, IL-8, GRO, CX3CL1, and CCL7 levels in culture supernatant at passage 5 and 7 in COPD patients. This effect was not observed in S-C patients.

### DISCUSSION

To the best of our knowledge this study shows, for the first time, that PGE₂ synthetized by senescent pulmonary fibroblasts in COPD can exert autocrine and paracrine effects inducing, reinforcing and propagating senescence and ensuing inflammation via EP₂ and EP₄ receptors, COX2-dependent ROS signaling and p53 signaling pathway. COPD fibroblasts were more sensitive to this phenomenon than smoker and non-smoker control cells.

EP₂ mRNA and protein expression were up-regulated in COPD non-senescent fibroblasts. This was particularly clear and different from both control groups and probably explains the particular sensitivity of COPD cells to PGE₂. This increase could be related to lung cancer, since fibroblasts were sampled from non-tumoral areas of lung excised for tumor pathology (Kreutzer M et al., Oncol Rep 2007; 18:497-501). However we carefully verified that our cells did not display any marker of cancer-associated fibroblasts. EP₂ overexpression in COPD fibroblasts could be related to DNA hypomethylation, since DNA methylation modulates negatively EP₂ expression (Huang S K et al., Am J Pathol 2010; 177:2245-2255), and aging and exposure to cigarette have been associated with global DNA hypomethylation (Smith I M et al., Int J Cancer 2007; 121:1724-1728; Bollati V et al., Mech Ageing Dev 2009; 130:234-239). In line with this epigenetic hypothesis, the increased COX2 expression in replicative senescent COPD cells could be related to a parallel decreased expression of miR 146a. Indeed, the expression of this miR, which negatively controls COX2 expression (Sato T et al., Am J Respir Crit Care Med 2010; 182:1020-1029), decreases with replicative senescence (Vasa-Nicotera M et al., Atherosclerosis 2011; 217:326-330). Alternatively, a positive feedback loop between PGE₂ and COX2 could explain the increased COX2 in senescent COPD fibroblasts since our results and other studies showed that PGE₂ can induce COX2 expression (Diaz-Munoz M D et al., Biochem J 2012; 443:451-461).

Although some fibroblast parameters were related to smoking per se and observed in S-C fibroblasts (e.g. the increase in PGE₂ and EP₄ expression at passage 3), the high expression of EP₂ at passage 3, and the increase in the majority of the senescence markers, and inflammation mediators (PGE₂, IL-8, GRO, CX3CL1, FGF2, TNF-α, RANTES) at passage 7 were only observed or significantly greater in COPD fibroblasts as compared to both control groups, and non reversible after smoking cessation, except for cumulative PDL at passage 7. Taking into consideration these data and the fact that control smokers had the same smoking history than COPD patients (whatever their current smoking status), the specificity of lung fibroblasts senescence in COPD is very likely. We have no explanation for the isolated reversibility of cumulative PDL in COPD fibroblasts after smoking cessation. However, the absence of reversibility of the majority of senescence and SASP markers in COPD fibroblasts is in agreement with a self-perpetuating process between senescence and inflammation (Acosta J C et al., Cell 2008; 133:1006-1018), which could explain the persistence of inflammation in COPD after smoking cessation (Rutgers S R et al., Chest 2000; 117:262S).

The effect of PGE₂, essentially involving either EP₂ or EP₄, is in line with previous studies showing that endogenous and/or exogenous PGE₂ decreased the proliferation of different cell types, including lung fibroblasts (Togo et al. Am J Respir Crit Care Med 2008; 178:248-260; Sato et al. Am J Respir Crit Care Med 2010; 182:1020-1029; Walker N M et al., Am J Respir Crit Care Med 2012; 185:77-84). We are confident in our results since we used several COX inhibitors and EP₂/EP₄ receptor agonists and antagonists of different chemical nature. A recent article showed that exogenous PGE₂ aggravated the replicative senescence of human dermal fibroblasts (Yang H H et al., Biogerontology 2011; 12:239-252). However, in that study the effect of PGE₂ was observed from 5 μM, a concentration almost 1500 times higher than in the present one, probably reflecting a pharmacological rather than a pathophysiological effect. Furthermore, no analysis of the SASP was performed in that study. Although it is difficult to compare in vitro and in vivo data, PGE₂ concentration in the secretome of COPD fibroblasts in the present study is in the same range as the one found in induced sputum from COPD patients (Chen Y et al., Respirology 2008; 13:1014-1021). This concentration of PGE₂ induced the secretion of inflammatory mediators by non-senescent COPD fibroblasts at levels close to those found in the SASP of COPD fibroblasts, stressing the pathophysiological significance of our results. Moreover, endogenous PGE₂ was directly involved in the autocrine and paracrine senescent effect of the SASP of COPD fibroblasts. However, since the SASP from senescent COPD fibroblasts contained cytokines capable of inducing and amplifying senescence, such as IL-6 and IL-8 (Acosta J C et al., Cell 2008; 133:1006-1018; Kojima H et al., Cell Cycle 2012; 11:730-739), one can argue that abrogation of senescence by COX2 inhibition was related to the absence of these factors and not to PGE₂. Although we cannot exclude this possibility completely, we think it unlikely, at least concerning IL-8, because no expression of CXCR2 mRNA was detected in COPD fibroblasts. Finally, since data from the literature shows that type 2 pneumocytes and macrophages can produce amounts of PGE₂ in the range of those produced by senescent COPD fibroblasts (Cao H et al., Anal Biochem 2008; 372:41-51; Mundandhara S D et al., Toxicol In Vitro 2006; 20:614-624; Mao J T et al., Clin Cancer Res 2003; 9:5835-5841), one cannot exclude a paracrine pro-senescent role on fibroblasts of PGE₂ synthesized by these neighboring cells.

The role of p53 on senescence induced by a single exposure to PGE₂ agrees with the well-established role of this protein in mediating accelerated senescence, whereas p16 is needed to completely arrest cell growth and drive the cell into replicative senescence (Zhang H. J Cell Physiol 2007; 210:567-574). Accordingly, although p16 was also increased by a single exposure to PGE₂, it was unmodified during p53 inhibition both in cells and mice, suggesting that it does not participate in the senescence induced by a single exposure to PGE₂. p53 was activated by an increased COX2-dependent ROS production, which is in line with previous results showing that sPLA₂, an extracellular phospholipase that cleaves phospholipids and yields arachidonic acid available as precursor for the production of prostaglandins, induces ROS in mice aorta via COX2 induction (van der Giet M et al., J Mol Med (Berl) 2010; 88:75-83). In contrast with the effect on senescence markers, the pro-inflammatory role of p53 after a single exposure to PGE₂ was unexpected since this protein has been repeatedly reported to be a negative regulator of the SASP (Coppe J P et al., Annu Rev Pathol 2010; 5:99-118). Only one study demonstrated opposite results showing that exposure of WI38 human lung fibroblasts to benzo[a]pyrene diol epoxide (BPDE) lead to IL-6, IL-8, and IL-1β secretion, through p53 and JNK pathways (Dreij K et al., Carcinogenesis 2010; 31:1149-1157). Interestingly, BPDE also induced COX2, pointing towards a particular effect of p53 activation via COX2 leading to a pro-inflammatory effect. Whatever the mechanism involved, our results demonstrate a new mechanism for the pro-inflammatory effects of PGE₂. This mechanism goes far beyond the fibroblast, since it was also demonstrated in total lung of wild type and p53−/− mice.

Repeated exposures of COPD fibroblasts to PGE₂ reproduced and enhanced the activation of the COX2/ROS pathway observed after a single exposure. Indeed, at passage 5 and 7 fibroblasts repeatedly exposed to PGE₂ showed not only an increased expression of HO-1 but also the activation of ATM, suggesting that in this case the level of ROS was enough to induce DNA damage (Banin S et al., Science 1998; 281:1674-1677). This phenomenon could explain the induction of some inflammatory mediators, such as IL-8 and GRO, which were not induced by a single PGE₂ application (Rodier F et al., Nat Cell Biol 2009; 11:973-979). In addition, PGE₂ enhanced the induction of p21 and p16 expression, without further decreasing telomeres length, thus showing a reinforcement of the pattern of replicative senescence occurring spontaneously in COPD cells. Such result is consistent with a PGE₂-induced oxidative signaling superimposed to the spontaneous process of replicative senescence, as demonstrated previously with other oxidative stimuli applied during replicative senescence (Frippiat C et al., Exp Gerontol 2000; 35:733-745; de Magalhaes J P et al., FEBS Lett 2002; 523:157-162). In line with these results the chronic treatment of COPD fibroblasts during passages 3 to 7 with COX2 inhibitors significantly decreased SA-β gal, p16, p21 and IL-6, IL-8, GRO, CX3CL1, MCP3 and MMP2 to levels close to those of control-smokers. In contrast, these inhibitors did not modify senescence and the SASP of control-smokers fibroblasts, stressing the particular sensitivity of COPD fibroblasts to the pro-senescent effect of the PGE₂/COX2 pathway.

In view of the present results, inhibition of the PGE₂ pathway could be an original approach to decrease senescence and inflammation in COPD. Since the prolonged use of COX2 inhibitors is linked to an increased risk of cardiovascular events, especially in COPD patients (Solomon D H et al., Arthritis Rheum 2008; 59:1097-1104), selective inhibition of other elements of the PGE₂ pathway, such as EP₂/EP₄ receptors, or mPGES-1 should constitute alternative original approaches.

### REFERENCES

Throughout this application, various references describe the state of the art to which this invention pertains. The disclosures of these references are hereby incorporated by reference into the present disclosure.
Wagena E J, van der Meer R M, Ostelo R J, Jacobs J E, and van Schayck C P. The efficacy of smoking cessation strategies in people with chronic obstructive pulmonary disease: results from a systematic review. Respir Med 2004; 98: 805-815.
Yoshida T and Tuder R M. Pathobiology of cigarette smoke-induced chronic obstructive pulmonary disease. Physiol Rev 2007; 87: 1047-1082.
Kim W Y and Sharpless N E. The regulation of INK4/ARF in cancer and aging. Cell 2006; 127: 265-275.
Coppe J P, Desprez P Y, Krtolica A, and Campisi J. The senescence-associated secretory phenotype: the dark side of tumor suppression. Annu Rev Pathol 2010; 5: 99-118.
Mannino D M, Buist A S. Global burden of copd: Risk factors, prevalence, and future trends. Lancet 2007; 370:765-773.
Muller K C, Welker L, Paasch K, et al. Lung fibroblasts from patients with emphysema show markers of senescence in vitro. Respir Res 2006; 7:32.
Nyunoya T, Monick M M, Klingelhutz A L, et al. Cigarette smoke induces cellular senescence via werner's syndrome protein down-regulation. Am J Respir Crit Care Med 2009; 179:279-287.
Campisi J. Senescent cells, tumor suppression, and organismal aging: Good citizens, bad neighbors. Cell 2005; 120:513-522.
Coppe J P, Desprez P Y, Krtolica A, et al. The senescence-associated secretory phenotype: The dark side of tumor suppression. Annu Rev Pathol 2010; 5:99-118.
Chen Y, Chen P, Hanaoka M, et al. Enhanced levels of prostaglandin e2 and matrix metalloproteinase-2 correlate with the severity of airflow limitation in stable copd. Respirology 2008; 13:1014-1021.
Togo S, Holz O, Liu X, et al. Lung fibroblast repair functions in patients with chronic obstructive pulmonary disease are altered by multiple mechanisms. Am J Respir Crit Care Med 2008; 178:248-260.
Sato T, Liu X, Nelson A, et al. Reduced mir-146a increases prostaglandin ein chronic obstructive pulmonary disease fibroblasts. Am J Respir Crit Care Med 2010; 182:1020-1029.
Normand J, Karasek M A. A method for the isolation and serial propagation of keratinocytes, endothelial cells, and fibroblasts from a single punch biopsy of human skin. In Vitro Cell Dev Biol Anim 1995; 31:447-455.
Lapperre T S, Sont J K, van Schadewijk A, et al. Smoking cessation and bronchial epithelial remodelling in copd: A cross-sectional study. Respir Res 2007; 8:85.
Savale L, Chaouat A, Bastuji-Garin S, et al. Shortened telomeres in circulating leukocytes of patients with chronic obstructive pulmonary disease. Am J Respir Crit Care Med 2009.
Rabe K F, Hurd S, Anzueto A, et al. Global strategy for the diagnosis, management, and prevention of chronic obstructive pulmonary disease: Gold executive summary. Am J Respir Crit Care Med 2007; 176:532-555.
Holz O, Zuhlke I, Jaksztat E, et al. Lung fibroblasts from patients with emphysema show a reduced proliferation rate in culture. Eur Respir J 2004; 24:575-579.
Dimri G P. What has senescence got to do with cancer? Cancer Cell 2005; 7:505-512.
Carter W, Narayanan P, Robinson J. Intracellular hydrogen peroxide and superoxide anion detection in endothelial cells. J Leukoc Biol 1994; 55:253-258.
Bankier A A, De Maertelaer V, Keyzer C, et al. Pulmonary emphysema: Subjective visual grading versus objective quantification with macroscopic morphometry and thin-section ct densitometry. Radiology 1999; 211:851-858.
Zdanov S, Bernard D, Debacq-Chainiaux F, et al. Normal or stress-induced fibroblast senescence involves cox-2 activity. Exp Cell Res 2007; 313:3046-3056.
Noureddine H, Gary-Bobo G, Alifano M, et al. Pulmonary artery smooth muscle cell senescence is a pathogenic mechanism for pulmonary hypertension in chronic lung disease. Circ Res; 109:543-553.
Kurz D J, Decary S, Hong Y, et al. Senescence-associated (beta)-galactosidase reflects an increase in lysosomal mass during replicative ageing of human endothelial cells. J Cell Sci 2000; 113 (Pt 20):3613-3622.
Woodward D F, Jones R L, Narumiya S. International union of basic and clinical pharmacology. Lxxxiii: Classification of prostanoid receptors, updating 15 years of progress. Pharmacol Rev 2011; 63:471-538.
Zhang H. Molecular signaling and genetic pathways of senescence: Its role in tumorigenesis and aging. J Cell Physiol 2007; 210:567-574.
Komarov P G, Komarova E A, Kondratov R V, et al. A chemical inhibitor of p53 that protects mice from the side effects of cancer therapy. Science 1999; 285:1733-1737.
Kreutzer M, Fauti T, Kaddatz K, et al. Specific components of prostanoid-signaling pathways are present in non-small cell lung cancer cells. Oncol Rep 2007; 18:497-501.
Huang S K, Fisher A S, Scruggs A M, et al. Hypermethylation of ptger2 confers prostaglandin e2 resistance in fibrotic fibroblasts from humans and mice. Am J Pathol 2010; 177:2245-2255.
Smith I M, Mydlarz W K, Mithani S K, et al. DNA global hypomethylation in squamous cell head and neck cancer associated with smoking, alcohol consumption and stage. Int J Cancer 2007; 121:1724-1728.
Bollati V, Schwartz J, Wright R, et al. Decline in genomic DNA methylation through aging in a cohort of elderly subjects. Mech Ageing Dev 2009; 130:234-239.
Vasa-Nicotera M, Chen H, Tucci P, et al. Mir-146a is modulated in human endothelial cell with aging. Atherosclerosis 2011; 217:326-330.
Diaz-Munoz M D, Osma-Garcia I C, Fresno M, et al. Involvement of pge2 and the camp signalling pathway in the up-regulation of cox-2 and mpges-1 expression in lps-activated macrophages. Biochem J 2012; 443:451-461.
Acosta J C, O'Loghlen A, Banito A, et al. Chemokine signaling via the cxcr2 receptor reinforces senescence. Cell 2008; 133:1006-1018.
Rutgers S R, Postma D S, ten Hacken N H, et al. Ongoing airway inflammation in patients with copd who do not currently smoke. Chest 2000; 117:262S.
Walker N M, Badri L N, Wadhwa A, et al. Prostaglandin e2 as an inhibitory modulator of fibrogenesis in human lung allografts. Am J Respir Crit Care Med 2012; 185:77-84.
Yang H H, Kim C, Jung B, et al. Involvement of igf binding protein 5 in prostaglandin e(2)-induced cellular senescence in human fibroblasts. Biogerontology 2011; 12:239-252.
Kojima H, Kunimoto H, Inoue T, et al. The stat3-igfbp5 axis is critical for il-6/gp130-induced premature senescence in human fibroblasts. Cell Cycle 2012; 11:730-739.
Cao H, Xiao L, Park G, et al. An improved lc-ms/ms method for the quantification of prostaglandins e(2) and d(2) production in biological fluids. Anal Biochem 2008; 372:41-51.
Mundandhara S D, Becker S, Madden M C. Effects of diesel exhaust particles on human alveolar macrophage ability to secrete inflammatory mediators in response to lipopolysaccharide. Toxicol In Vitro 2006; 20:614-624.
Mao J T, Roth M D, Serio K J, et al. Celecoxib modulates the capacity for prostaglandin e2 and interleukin-10 production in alveolar macrophages from active smokers. Clin Cancer Res 2003; 9:5835-5841.
van der Giet M, Tolle M, Pratico D, et al. Increased type iia secretory phospholipase a(2) expression contributes to oxidative stress in end-stage renal disease. J Mol Med (Berl) 2010; 88:75-83.
Dreij K, Rhrissorrakrai K, Gunsalus K C, et al. Benzo[a]pyrene diol epoxide stimulates an inflammatory response in normal human lung fibroblasts through a p53 and jnk mediated pathway. Carcinogenesis 2010; 31:1149-1157.
Banin S, Moyal L, Shieh S, et al. Enhanced phosphorylation of p53 by atm in response to DNA damage. Science 1998; 281:1674-1677.
Rodier F, Coppe J P, Patil C K, et al. Persistent DNA damage signalling triggers senescence-associated inflammatory cytokine secretion. Nat Cell Biol 2009; 11:973-979.
Frippiat C, Chen Q M, Remade J, et al. Cell cycle regulation in h(2)o(2)-induced premature senescence of human diploid fibroblasts and regulatory control exerted by the papilloma virus e6 and e7 proteins. Exp Gerontol 2000; 35:733-745.
de Magalhaes J P, Chainiaux F, Remade J, et al. Stress-induced premature senescence in bj and htert-bj1 human foreskin fibroblasts. FEBS Lett 2002; 523:157-162.
Solomon D H, Glynn R J, Rothman K J, et al. Subgroup analyses to determine cardiovascular risk associated with nonsteroidal antiinflammatory drugs and coxibs in specific patient groups. Arthritis Rheum 2008; 59:1097-1104.

1. A method for preventing or treating senescence and inflammation in COPD in a subject in need thereof, comprising the step of administering to said subject a compound which is selected from the group consisting of PGE2-receptor antagonists, PGE2-receptor expression inhibitors, COX-2 inhibitors, COX-2 expression inhibitors, prostaglandin E2 synthase inhibitors or prostaglandin E2 synthase expression inhibitors.

2. The method according to claim 1 wherein said PGE2-receptor antagonist is selected from EP2 antagonists or EP4 antagonists.

3. The method according to claim 1 wherein said PGE2-receptor antagonist is selected from AH-6809 or GW627368X and said COX-2 inhibitor is selected from indomethacin or celecoxib.

4. A method of screening a candidate compound for use as a drug for the prevention or treatment of senescence and inflammation in COPD in a subject in need thereof, wherein the method comprises the steps of:
providing a PGE2, PGE2-receptor, COX-2, prostaglandin E2 synthase, providing a cell, tissue sample or organism expressing the PGE2-receptor,
providing a candidate compound such as small organic molecule, prostaglandin analogues, antibodies, peptide or polypeptide,
measuring the activity of the PGE2-receptor, COX-2 or prostaglandin E2 synthase,
and selecting positively candidate compounds that block PGE2-receptor, inhibit PGE2-receptor expression, inhibit COX-2 activity, inhibit COX-2 expression, inhibit prostaglandin E2 synthase activity or inhibit prostaglandin E2 synthase expression.
